(19) United States
(12) Patent Application Publication
(10) Pub. No.: US 2013/0287745 A1
(43) Pub. Date: Oct. 31, 2013

(54) COMPOSITIONS AND METHODS TO STIMULATE VASCULAR STRUCTURE FORMATION

(71) Applicants: Keith Leonard March, Carmel, IN (US); Brian Johnstone, Indianapolis, IN (US); Dmitry O. Traktuev, Indianapolis, IN (US)

(72) Inventors: Keith Leonard March, Carmel, IN (US); Brian Johnstone, Indianapolis, IN (US); Dmitry O. Traktuev, Indianapolis, IN (US)

(21) Appl. No.: 13/930,777

(22) Filed: Jun. 28, 2013

Related U.S. Application Data
(63) Continuation of application No. 12/526,656, filed on Feb. 15, 2010.

(51) Int. Cl. A61K 35/12

(52) U.S. Cl. CPC A61K 35/35
USPC 424/93.7

(57) ABSTRACT

Cell based compositions and methods are provided for inducing the formation of vascular structures in a warm blooded vertebrate. In one embodiment the composition comprises purified endothelial progenitor cells and adipose stromal cells and the method of stimulating the formation of vascular structures comprises the steps of implanting the composition in a host organism.

### PRIORITY

The present application is related to, claims the priority benefit of, and is a U.S. continuation application of, U.S. Ser. No. 12/526,656, filed Aug. 11, 2009, which is related to, claims the priority benefit of, and is a U.S. §371 application of, International Application Serial No. PCT/US08/53992, filed Feb. 14, 2008, which is related to, and claims the priority benefit of, U.S. Ser. No. 60/889,852, filed Feb. 14, 2007.

### BACKGROUND

Rapid induction and maintenance of blood flow through new vascular networks is essential for successfully treating ischemic tissues and maintaining the function of engineered neo-organs. A general requirement for preserving viable tissues at the border of an ischemic zone, or within a regenerating region, is that a vascular bed is assembled or expanded rapidly and extensively to ensure adequate perfusion within the tissues. Also important to the success of such applications is the ability of any network to anastomose as promptly as possible with the vessels of immediately adjacent tissues, which will provide the blood flow.

Cell-based revascularization therapies have been recently extended to clinical studies for testing in patients that suffer from various ischemic diseases, particularly those diseases involving the heart and limbs. Most studies have been conducted with autologous cells due to considerations of immunotolerance. These studies have employed a variety of progenitor and stem cell types, commonly isolated from bone marrow and skeletal muscle delivered to patients with myocardial infarction, heart failure, peripheral vascular disease and muscular dystrophy. Despite the fact that accumulating data and recent meta-analyses strongly support the hypothesis that certain progenitor and stem cells have a high potential for promoting tissue revascularization and functional recovery, technical and practical limitations exist due to the invasive methods of harvest and low abundance, which may limit adoption of therapies employing several cell types.

As disclosed herein, adipose stromal cells (ASCs) are a population of pluripotent mesenchymal cells which are readily available in large numbers from adipose tissue. These cells are predominantly associated with blood vessels in vivo, and have been discovered to be phenotypically and functionally equivalent to pericytes associated with microvessels. Endothelial progenitor cells (EPCs) have been studied extensively over the past decade since their original isolation from adult peripheral blood and, later from bone marrow, umbilical cord blood, and vessel wall. Umbilical cord blood (UCB) contains a population of EPC with a particularly high proliferative potential, referred to herein as endothelial colony forming cells (ECFCs).

Recently ECFCs have been found to form functional vessels in vivo when implanted in a matrix in mice (Ingram, D. A. et al., Stem cells (Dayton, Ohio) 25, 297-304 (2007). While the presence of blood cells within the capillary networks formed by such human EPCs confirmed anastomoses with host vasculature, the neovessels were limited in frequency and size (Au, P. et al., Blood (2007). This extended a prior observation for implants containing untransformed adult endothelial cells, which yielded vessels characterized as narrow-caliber with single-layer walls (Schechner, J. S. et al., Proc Natl Acad Sci USA 97, 9191-9196 (2000). In the latter study, forced overexpression of bcl-2 in the endothelial cells conferred the ability to form larger-caliber vessels with thicker walls, presumably as a consequence of repressing endothelial apoptosis as well as augmenting recruitment of mesenchymal cells from the murine host. With non-transformed endothelial cells, the failure to establish stable, mature vasculature may be due to prolonged absence of a stabilizing layer of mural cells such as pericytes or smooth muscle cells.

Although EPCs secrete multiple angiogenic factors that attract perivascular cells, conditions within an implanted composition may not attract sufficient host mural cells within an appropriate timeframe to promote stability of neovasculature before competing forces act to disassemble the vessels. Applicants recognized that ASCs, which possess properties of pericytes might be an ideal and practical cell type to co-implant with endothelial cells, for the immediate support and stabilization of vessel formation initiated by endothelial cells in ischemic tissues. The ease with which large numbers of autologous ASCs can be harvested following minimally invasive liposuction supports their practical utility in a range of therapeutic approaches. As disclosed herein human ASCs in combination with EPCs stimulate vasculogenesis to form stable functional vasculature in vivo when the cells are co-implanted, leading to active network remodeling, inosculation with host vasculature, and rapid provision of blood flow.

### SUMMARY

As disclosed herein a composition comprising a mixture of purified endothelial cells and purified adipose stromal cells is provided for stimulating the production of functional vascular networks. In accordance with one embodiment the compositions comprise adipose stromal cells and endothelial progenitor cells, optionally combined with a biocompatible polymer. In one embodiment the biocompatible polymer is a protein (such as collagen) or a peptide. The purified adipose stromal cells and endothelial progenitor cells are typically primary cells that are purified from mammalian tissues, including for example, from adipose tissue and umbilical cord blood, respectively. In one embodiment the cells are held within a collagen/fibronectin matrix.

The present disclosure further describes a method of creating a vessel network. The method comprises the steps of mixing a purified population of endothelial cells with a purified population of adipose stromal cells to produce a mixture of cells, and incubating the mixture of cells under conditions conducive for the growth of said cells, resulting in the formation of a network of vessels.

The present disclosure further encompasses a kit for inducing the formation of vascular networks. The kit comprises a purified population of endothelial cells and a purified population of adipose stromal cells. The kit may comprise additional components for use in expanding the initial populations of endothelial or stromal cells, as well as components for administering the cells to a patient. In one embodiment the kit further comprises components for forming a biocompatible matrix to be used in conjunction with the cells.

### BRIEF DESCRIPTION OF THE DRAWINGS

FIG. 1 is a bar graph depicting the data generated from macroscopic and microscopic examination of implants. Collagen/fibronectin matrices containing either EPCs alone, ASCs alone, or a combination of ASCs and EPCs (at a 1:4 ratio) were implanted subcutaneously in NOD/SCID mice (N=6-8/each type of implant), and harvested after 2 weeks. Histochemical staining of sections with hematoxylin and eosin (H&E) was performed to identify vessels for subsequent quantitative analysis. Implants were categorized according to vessel presence and morphology, demonstrating a clear enhancement in the frequency of multilayer vascularization by the admixture of cell types.

FIGS. 2A-2C are bar graphs representing immunohistochemical evaluation of vascular structures formed in implants, revealing incorporated human endothelial cells. Thin sections of formalin fixed, paraffin-embedded implants were probed with either human-specific antibodies to the endothelial cell marker CD3I, or antibodies to the mural cell marker smooth muscle α-actin (α-SMA) and stained with hematoxylin to visualize nuclei. Multiple locations in the matrices were obtained and analyzed for density of CD3I (FIG. 2A) and α-SMA (FIG. 2B) staining vessels, as well as the distribution of vessels diameters (FIG. 2C), in a blinded fashion using Image J analysis software. The number of implants used for analysis were 10 (EPC), 7 (ASC), and 21 (Both). (***, p<0.001).

FIGS. 3A & 3B present data showing an evaluation of functional vessel density and dynamics of network formation in implants containing both ASCs and EPCs. The density of vessels containing donor-derived endothelium recognized by anti-human CD3I antibody, that anastomosed with host vasculature as indicated by the presence of red blood cells (RBC5) in the lumens, was quantitated in sections of fixed, embedded implants probed with antibodies to human CD3I and imaged at 200× magnification (see FIG. 3A). Multiple implants from each group (EPC5, n=13; ASCs, n=7; and both, n=24) were analyzed at 14 days post-implant, and the data are expressed as vessel area density. Ultrasound imaging was performed on a subset of sedated animals to demonstrate intra-implant blood flow in vivo using echogenic microbubbles. The earlier temporal progression of vessel formation was determined by performing histological analyses on matrices containing both ASC and EPC that had been implanted for 2, 4 and 6 days; then fixed, embedded, probed with antibodies against human CD3I, and stained with hematoxylin (n=4 for each time). The density of the RBC-containing CD3I-positive vessels was quantitated at these latter timepoints, and is shown (FIG. 3B). (*, p<0.05); ***p<0.001).

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms. For example, as used herein the term “treating ischemic tissues” will refer in general to any increase in blood flow to the ischemic tissues.

As used herein an “effective” amount or a “therapeutically effective amount” of a composition refers to a nontoxic but sufficient amount of the composition to provide the desired effect. For example one desired effect would be the production of sufficient neovasculature to prevent or treat ischemic tissue. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as subcutaneous, intramuscular, intraspinal, or intravenous.

As used herein the term “adipose stromal cells” refers to pluripotent stem cells that recovered from adipose tissue. Typically the cells express at least one cell marker selected from the group CD14Oa, CD14Ob and NG2.

As used herein the term “endothelial progenitor cell” refers to committed stem cells that have the ability to differentiate into endothelial cells, the cells that make up the lining of blood vessels. Typically endothelial progenitor cells express at least one cell marker selected from the group consisting of CD34, CD133, CD31, VE-cadherin, VEGFR2, CD31, CD45, Tie-2 and c-Kit. In one embodiment the endothelial progenitor cells express the cell markers CD133 and CD34.

As used herein, the term “endothelial colony forming cells (ECFCs)” refers to endothelial progenitor cells that are capable of proliferation and colony formation upon culturing the cells in vitro.

As used herein the term “functional blood vessels” or “functional vascular network refers to vessels/vessel networks that are stable, multi-cell layered and are connected with host vasculature and carry erythrocytes in their lumen.

As used herein, the term “purified” and like terms relate to an enrichment of a selected compound or selected cells relative to other components or cells normally associated with the selected compound or selected cells in a native environment. The term “purified” does not necessarily indicate that complete purity of the particular cells/compound has been achieved during the process. For example a purified adipose stromal cell comprises adipose stromal cells substantially free of adipocytes, endothelial cells and blood derived cells.

As used herein the term “native” in reference to a cell population is intended to indicate that the genetic components of the cell have not been altered by human directed recombinant nucleic acid manipulation. The term is not intended to exclude a population of cells that have been purified, or subjected to other non-recombinant nucleic acid manipulations.

As used herein the term “patient” without further designation is intended to encompass any warm blooded vertebrate domesticated animal (including for example, but not limited to livestock, horses, cats, dogs and other pets) and humans.

### EMBODIMENTS

Formation and remodeling of vascular networks are critical in both the development of normal tissues and their response to injury. Engineering of tissue constructs with thickness greater than accommodated by gas or nutrient diffusion will also require practical means for the provision of vascular components that invest the constructs and provide blood flow as promptly as possible upon implantation. In addition, the local augmentation of vascular network development has been an important goal for therapy of ischemic disorders such as myocardial infarction and peripheral vascular diseases. As disclosed herein two readily available, genetically unmodified primary human cell types, when combined, exert a synergistic effect that enhances the de novo formation of vascular networks.

Endothelial progenitor cells by themselves demonstrate a limited ability to form vasculature structures de novo in mice, but these structures are limited in number and persistence. As disclosed herein, applicants have discovered that the combination of such cells with an additional supporting population of cells, such as adipose stromal cells, produces a synergistic effect that leads to the de novo production of functional blood vessels. In accordance with one embodiment a composition is provided comprising a purified population of endothelial cells and a purified population of pericytes and/or adipose stromal cells (ASCs). In one embodiment the endothelial cells are progenitor endothelial cells (EPCs) and in a further embodiment the endothelial cells are colony forming cells. The composition comprising the purified ASCs and EPCs are administered to a warm blooded vertebrate to provide a synergistic effect resulting in de novo formation of vascular networks. In one embodiment the host organism receiving the composition is a mammal and in one embodiment the mammal is a human.

The endothelial cells used in accordance with the present disclosure may be isolated from any part of the vascular tree, as they comprise the lining of blood vessels. Accordingly, endothelial cells are present in large and small veins and arteries, from capillaries, or from specialized vascular areas such as the umbilical vein of newborns, blood vessels in the brain, or from vascularized solid tumors. Endothelial progenitor cells are bone marrow-derived cells that circulate in the blood and have the ability to differentiate into endothelial cells. Endothelial progenitor cells (EPCs) can be isolated from adult peripheral blood, bone marrow, umbilical cord blood, and vessel walls. Umbilical cord blood (UCB) contains a population of EPC with a particularly high proliferative potential, and provides a source for endothelial colony forming cells (ECFCs).

Purification of endothelial progenitor cells can be conducted using standard procedures known to those skilled in the art. The partially or completely purified endothelial cells may then be directly combined with adipose stromal cells, or alternatively, the purified endothelial cells can be first cultured in vitro, in media that will support the growth of fibroblasts, for a period of between eight hours to up to five cell passages prior to combination with the adipose stromal cells.

The adipose stromal cells used in accordance with the present disclosure may be isolated from adipose tissues (i.e. any fat tissue). The source adipose tissue may be brown or white adipose tissue. In one embodiment, the adipose stromal cells are purified from subcutaneous white adipose tissue. The adipose tissue may be from any organism having fat tissue, however typically the adipose tissue is mammalian, and in one embodiment the adipose tissue is human. A convenient source of human adipose tissue is material recovered from liposuction procedures, however, the source of adipose tissue or the method of isolation of adipose tissue is not critical to the invention.

In accordance with one embodiment, adipose stromal cells are purified from their source material by treating adipose tissue so that the stromal cells are dissociated from each other and from other cell types, and precipitated blood components are removed. Typically, dissociation into single viable cells may be achieved by treating adipose tissue with proteolytic enzymes, such as collagenase and/or trypsin, and with agents that chelate Ca²⁺. Stromal cells may then be partially or completely purified by a variety of means known to those skilled in the art, such as differential centrifugation, fluorescence-activated cell sorting, affinity chromatography, and the like. The partially or completely purified stromal cells may then be directly combined with endothelial cells, or alternatively, the purified stromal cells are first cultured in vitro, in media that will support the growth of fibroblasts, for a period of between eight hours to up to five cell passages prior to combination with the endothelial cells.

In one embodiment the adipose stromal cells are native cells purified from the tissues of same patient that they will be ultimately be administered to (i.e., autologous transplantation), albeit in combination with a purified population of native endothelial cells. In one embodiment both the adipose stromal cells and the endothelial cells are purified from the tissues of same patient that they will ultimately be administered (i.e., autologous transplantation). In accordance with one embodiment the purified adipose stromal cells express the cell markers CD14Oa, CD14Ob, and NG2, and in a further embodiment the endothelial progenitor cell comprise cells that express the cell markers CD133 and/or CD34. In accordance with one embodiment the purified endothelial cells and purified adipose stromal cells are both native cell populations. In another embodiment the purified endothelial cells and purified adipose stromal cells are further manipulated to express recombinant gene products that assist in the formation and maintenance of vascular structures. Such gene products include growth factors such as VEGF, HGF, and angiopoietin-1, FBS, and EGM-2.

The ratio of endothelial cells to stromal cells can be varied, however the endothelial cells will typically out number the stromal cells by at least 2:1, more typically by much greater margins of 4:1, 5:1, 8:1, 10:1 and 20:1. In one embodiment the cell mixture comprises about a 4:1 ratio of endothelial progenitor cells to adipose stromal cells. The total cells administered to the patient will vary base on the method of administration and the site of administration. Typically the cells are administered at a cell density of about 1×10⁵ to about 1×10⁷ cells/ml, or in one embodiment about 5×10⁵ to about 5×10⁶ cells/ml.

In accordance with one embodiment the purified cells (e.g., ASCs and EPCs) are combined with a biocompatible polymer. Biocompatible polymers suitable for use with the cell compositions disclosed herein include, but are not limited to proteins (e.g. collagen), peptides, polyglycol acid (PGA), polylactic acid (PLA) or a co-polymer of PGA and PLA, alkyl celluloses, hydroxyalky methyl celluloses, hyaluronic acid, sodium chondroitin sulfate, polyacrylic acid, polyacrylamide, polycyanolacrylates, methyl methacrylate polymers, 2-hydroxyethyl methacrylate polymers, cyclodextrin, polydextrose, dextran, gelatin, polygalacturonic acid, polyvinyl alcohol, polyvinyl pyrrolidone, polyalkylene glycols, and polyethylene oxide. In accordance with one embodiment the biocompatible polymer are biodegradable polymers, and in accordance with one embodiment the cell composition further comprises collagen and fibronectin, and more particularly type I collagen.

In accordance with one embodiment the polymers are assembled into a matrix that surrounds and entraps the cells. For example the cells can be suspended or embedded within a biocompatible matrix that at least temporarily restricts the migration of the cells from the matrix. In one embodiment the matrix is a biodegradable matrix. In one embodiment a collagen/fibronectin matrix is employed to provide a supportive scaffold within which the ASCs and EPCs could interact without leaking from the site of implantation. However, it is anticipated that cell delivery can be accomplished in a range of matrices that may assist both in restricting redistribution and augmenting survival. Such compositions are anticipated to be particularly useful in ischemic environments which may be hostile to implanted cells. Biocompatible matrices suitable for use in the present invention are known to those skilled in the art and include, but are not limited to those comprising hydrogels (including for example PuraMatrix™ Peptide Hydrogel; Becton, Dickinson, Inc), alginate, MATRIGEL™ (BD Biosciences, Sparks, Md.), collagen, peptides, polyglycol acid (PGA), polylactic acid (PLA), co-polymers of PGA and PLA, poly(ether ester), polyethylene glycol (PEG), or block copolymers of PEG and poly(butylene terephthalate) materials.

In accordance with one embodiment the cells are suspended in a PuraMatrix™ Peptide Hydrogel (Becton, Dickinson, Inc) matrix. PuraMatrix™ Peptide Hydrogel is a synthetic matrix that is used to create defined three dimensional (3D) microenvironments for a variety of cell culture experiments. In one embodiment the matrix is further combined with additional bioactive molecules (e.g., growth factors, extracellular matrix (ECM) proteins, and/or other molecules). PuraMatrix™ Peptide Hydrogel consists of standard amino acids (1% w/v) and 99% water. Under physiological conditions, the peptide component of PuraMatrix™ Peptide Hydrogel self-assembles into a 3D hydrogel that exhibits a nanometer scale fibrous structure with an average pore size of 50-200 nm. The hydrogel is readily formed in a culture dish, plate, or cell culture insert.

In another embodiment a biodegradable matrix comprising collagen, or a mixture of collagen and fibronectin, is provided. In a further embodiment the cell composition comprises a collagen matrix, wherein the collagen matrix comprises about 1.0 to about 2.0 mg/ml collagen type I, and about 50 to about 150 ng/ml human fibronectin. In a further embodiment the cell compositions further comprise an exogenous source of FBS, and EGM-2. In one embodiment, the biodegradable matrix has a half-life of about 1 to 60 days, or alternatively, a half-life of about 14 to 30 days.

In accordance with one embodiment the cell composition is maintained in an injectable form. For example, the cell composition may comprise a mixture of endothelial cells and adipose stromal cells and a pharmaceutically acceptable carrier, wherein the mixture of cells is suspended in said carrier. In one embodiment a composition comprising the cells and a pharmaceutically acceptable carrier is injected into a patient at a site in need of enhanced vascularization. In one embodiment the cells are suspended in a biodegradable matrix and the composition is injected near, or into, tissues in need of enhanced vascularization, include for example ischemic tissue.

The present endothelial and adipose stromal cell compositions can be used to stimulate the formation of de novo vascular structures in vitro or in vivo. In accordance with one embodiment a method of creating a vessel network comprises the steps of mixing a purified population of endothelial cells with a purified population of adipose stromal cells to produce a mixture of cells. The mixture of cells is then incubated under conditions conducive for growth of said cells. Conditions suitable for the growth of endothelial cells and adipose stromal cells in vitro are known to those skilled in the art. Alternatively the incubating conditions can be the in vivo environment of a patient after the cell composition is injected/implanted in the patient. The growth of the endothelial and adipose stromal cells in each others presence results in the formation of a network of vessels. More particularly, the vessels formed are multi-layered, comprising an inner endothelial layer surrounded by an outer layer of α-SMA⁺ cells.

One advantage of the present invention relates to the ease of obtaining ASCs and blood-derived EPCs from human tissues. Moreover, both types of cells possess high proliferative activity in culture, sufficient to rapidly amplify initial cell preparations if required. ASCs represent a readily accessible autologous population of cells expressing multiple markers (CD14Oa, CD14Ob, NG2) and physiological characteristics of pericytes. In vivo evaluation of compositions comprising ASC and EPC cells reveals that this combination of cells produces a remarkably dense and stable assembly, demonstrating the ability of ASC to behave as pericytes in vivo. An important effect of ASCs on endothelial cells involves abrogation of the marked apoptosis present in implants containing only endothelial cells. This is also consistent with previously reported findings that factors released from ASCs can protect endothelial cells from apoptosis in vitro (Rebman, J. et al. Circulation 109, 1292-1298 (2004), as well as stabilize EC cord formation on MATRIGEL™ in vitro (Traktuev, D. et al. A Population of Multipotent CD34-Positive Adipose Stromal Cells Share Pericyte and Mesenchymal Surface Markers, Reside in a Periendothelial Location, and Stabilize Endothelial Networks. Circ Res (2007).

Several molecular mechanisms may be involved in these effects of ASCs on endothelial cells, including the secretion by ASCs of diffusible pro-angiogenic and anti-apoptotic factors (including VEGF, HGF, and angiopoietin-1), as well as direct contact with newly forming endothelial tubes. Given this apparent role of ASCs in supporting endothelial cell survival during the process of vasculogenesis, it was of interest to ascertain whether endothelial cells play a complementary role in modulating ASC behavior via factors secreted by endothelial cells. PDGF-BB is a key factor secreted by endothelial cells and EPCs. The result of local blockade of PDGF-BB function by a neutralizing antibody to PDGF was a complete interruption of vasculogenesis, suggesting a role for diffusible signaling from endothelial cells to ASCs in this system. This result also provides further support for the notion that ASCs function as pericytes, against which PDGF blockade has recently been found to play an important role in cancer therapy, reducing tumor growth via inhibition of endogenous pericytes investing tumor vasculature (Bergers, et al., The Journal of clinical investigation 111, 1287-1295 (2003).

Both in the context of an engineered implant as well as for therapeutic augmentation of tissue perfusion, timely provision of functional circulation is essential. Accordingly, one embodiment disclosed herein is directed to a method of enhancing the de novo production of localized functional vascular networks in vivo. In one embodiment a composition comprising a purified population of EPCs and a purified population of ASCs is placed in contact with a site in need of improved vascularization. In one embodiment the composition is injected or implanted at the desired site. In one embodiment the composition further comprises a matrix that impedes the mobility of the cells at least temporarily after injection/implantation. In one embodiment the cells are purified from tissues of the same individual to receive the purified EPC/ASC cell composition. The purified cells can be immediately injected/implanted after the purification steps or alternatively the cells can be cultured either separately, or co-cultured, in vitro prior to being administered to the patient.

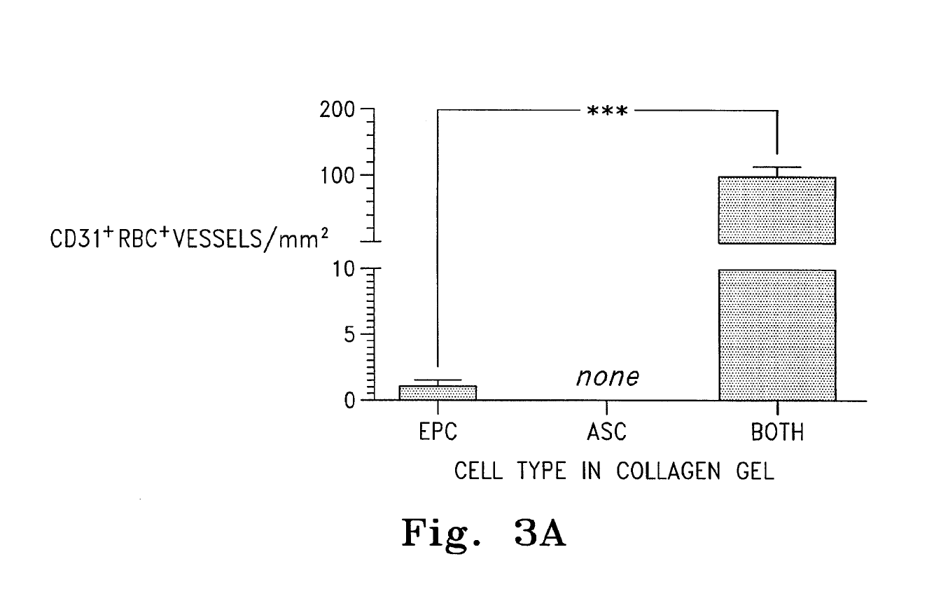

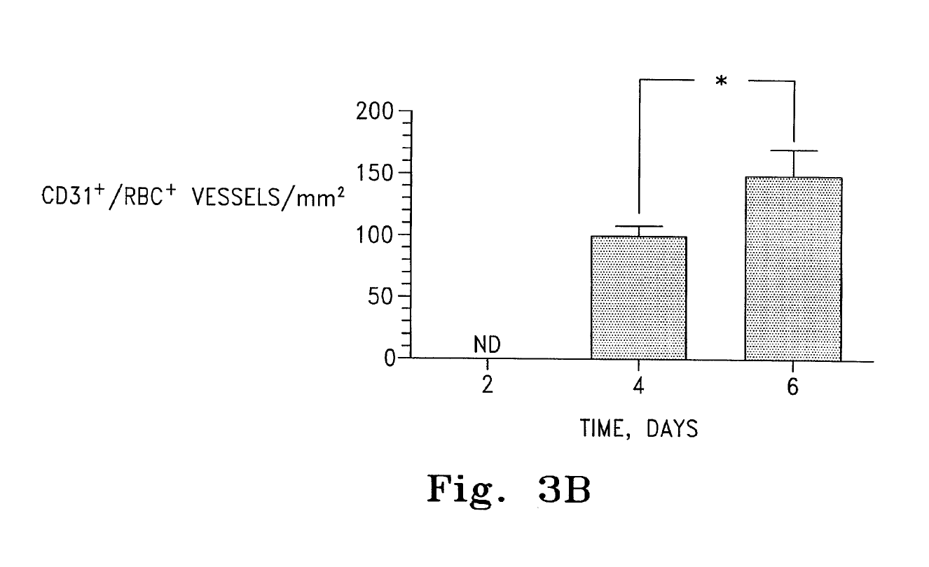

Applicants have observed that the human donor-derived vessels have routinely established communication with the host circulation by day 4 following implantation of the EPC/ASC cell composition (see Examples, FIG. 3B). Analysis of cell cycling revealed active proliferation of both vascular layers in the implants, suggesting involvement of proliferation as well as assembly and host vessel inosculation. The extent to which the input cells are initially capable of expansion following implantation is not clear, but the stabilization of the vascular density between days 7 and 14 post-implant in the collagen gels suggests intrinsic mechanisms controlling proliferation, concurrently with vascular remodeling in the context of flow.

In addition to translational utility for tissue engineering and vascular augmentation using clinically practical cells, the chimeric mouse/human system disclosed in the examples will also have utility for dissecting mechanisms that govern proliferation, lumen assembly, donor-host interaction, branching, and density regulation of human vasculature, by providing the opportunity to independently manipulate human endothelial and mural cells prior to the onset of vasculogenesis. In accordance with one embodiment compositions comprising EPC and ASC can be used to screen for bioactive compounds and pharmaceutical compositions that affect, either positively or negatively angiogenesis. In accordance with one embodiment the method comprises co-culturing the EPC and ASC cells under conditions suitable for the formation of functional vascular networks in both the presence and absence of a compound of interest to screen for compounds that stimulate or inhibit the formation of vascular structures. Alternatively, the composition comprising the EPC and ASC cells can be injected or implanted into an animal and the animal can be administered a pharmaceutical composition to determine the pharmaceutical's effect on vasculogenesis.

In a parallel manner, the EPC and ASC “two-cell system” also provides a means for evaluating the role of matrix in vasculogenesis. In one embodiment a collagen/fibronectin matrix is used to provide a supportive scaffold within which the ASCs and EPCs can interact without leaking from the site of implantation. However, the role of the matrix in vasculogenesis can be investigated by the selection of other biocompatible matrices that are known to those skilled in the art. It is anticipated that such matrices will provide an optimal delivery vehicle (assisting both in restricting redistribution and augmenting survival) in some environments, particularly in ischemic environments which may be hostile to implanted cells.

In addition to delivery of the cells within an exogenous matrix, the results provided in the examples show that EPC and ASC compositions are capable of assembly into vascular structures both in the region of ischemic tissue (myocardium) as well as in a non-ischemic tissue (such as the mouse ear).

The ready availability of ASCs and EPCs from clinically feasible sources, and their simple, well-defined preparation provide attractive features for utility of the system.

Additionally, ASCs can be successfully harvested with yields which eliminate the need for subsequent expansion of the recovered cells. One rich source of EPCs is umbilical cord blood which has demonstrated the ability to proliferate extensively.

In accordance with one embodiment a method of inducing the formation of a functional vascular network in a patient is provided. Advantageously, the vessels formed by the methods disclosed herein are multilayered vessels comprising an inner endothelial layer surrounded by an outer layer of α-SMA⁺ cells. In accordance with one embodiment the method allows for the formation a new network of vessels (at a density of 92.5±16.2 per mm²), wherein over 70% of CD31⁺ vessels formed in vivo are functional and blood-filled. In accordance with one embodiment, the vascular network formed in accordance with the disclosed method has greater than 90% of the αSMA⁺ vessels having a vessel diameter of at least 5 pun. In one embodiment the density of αSMA⁺ vessels formed de novo is greater than 100 vessels/mm², and more particularly the density of αSMA⁺ vessels having a diameter of at least 10 μm is greater than 60 vessels/mm², with the density of αSMA⁺ vessels having a diameter of at least 15 μm being greater than 20 vessels/mm². In one embodiment the method comprises placing the endothelial/adipose cell compositions into a warm blooded vertebrate at the site where de novo formation of a functional vascular network is desired. In one embodiment the purified endothelial cells and purified adipose stromal cells are both native autologous cell populations that were purified from the patient that receives the endothelial/adipose cell composition. In one embodiment the endothelial/adipose cell composition is injected at the desired site, and in an alternative embodiment the cell composition is surgically implanted in the patient.

In accordance with one embodiment a kit is provided for forming functional vascular networks. In one embodiment the kit for inducing the formation of vascular networks comprises a purified population of endothelial cells and a purified adipose stromal cells. The kit may further comprise additional components for the in vitro culturing of the cells as well as instructional material and sterile labware. In accordance with one embodiment the kit further comprises a biocompatible polymer, including but not limited to collagen, fibronectin, polyglycol acid (PGA), polylactic acid (PLA) or a co-polymer of PGA and PLA. In one embodiment the endothelial cells are endothelial progenitor cells and the kit comprises a container comprising collagen and a container comprising fibronectin. In further embodiment the kit comprises growth factors including for example, FBS, and EGM-2.

### Example 1

Mixture of endothelial cells and adipose stromal cells and implantation into a host provides a synergistic effect leading to the formation of functional blood vessels.

Methods

Mononuclear Cells Isolation

Peripheral blood was collected from umbilical cord blood of healthy newborns (38-40 weeks gestational age) as described in Ingram, D. A., et al., Blood, 2004. 104(9): p. 2752-60. Mononuclear cells (MNCs) were isolated from blood samples by gradient centrifugation over Histopaque 1077 (ICN) and washed with EBM-2 medium (Cambrex, Baltimore, Md.) supplemented with 10% FBS (Hyclone, Logan, Utah), 100 units/ml penicillin, 100 pg/ml streptomycin and 0.25 μg/ml of amphotericin B (EGM-2/F medium; Invitrogen, Carlsbad, Calif.) as described in Ingram, D. A., et al., Blood, 2004. 104(9): p. 2752-60.

Isolation and Culture of EPCs

Isolated MNC were resuspended in EGM-2/F. Cells were plated into six well tissue culture plates (5×10⁷ cells/well) pre-coated with type I rat tail collagen (BD Biosciences, San Diego, Calif.) and incubated at 37° C., 5% CO₂ as described in Ingram, D. A., et al., Blood, 2004. 104(9): p. 2752-60. Medium was changed daily for seven days and then every other day until first passage. Once confluent, EPCs were trypsinized, resuspended in EGM-2/F medium, and plated onto 75 cm² tissue culture flasks coated with type I rat tail collagen. EPC monolayers were passaged after becoming 90-100% confluent and used after four to six passages.

Isolation and Culture of Human Adipose Stromal Cells (hASCs)

Human subcutaneous adipose tissue samples (N=10), obtained from lipoaspiration/liposuction procedures were digested in a 1 mg/ml Collagenase Type I solution (Worthington Biochemical, Lakewood, N.J.), supplemented with 10% FBS, 100 units/ml penicillin and 100 pg/ml streptomycin, under gentle agitation for 2 hours at 37° C. and centrifuged at 300 g for 8 minutes to separate the stromal cell fraction (pellet) from adipocytes. The cell pellet was resuspended in DMEM/F12 containing 10% FBS (Hyclone, Logan, Utah) filtered through 250 μm Nitex filters (Sefar America Inc., Kansas City, Mo.) and centrifuged at 300 g for 8 minutes. To eliminate erythrocyte contamination the cell pellet was treated with red cell lysis buffer (154 mM NH₄Cl, 10 mM KHCO₃, 0.1 mM EDTA) for 10 minutes. The final cell pellet was resuspended and cultured in EGM2-MV (Cambrex, Baltimore, Md.). ASC monolayers were passaged after becoming 60-80% confluent and used after 3-6 passages.

Xenograft EPC Transplantation

Cellularized gel implants were cast as previously described with minor modifications (see Schechner, J. S., et al., Proc Natl Acad Sci USA, 2000. 97(16): p. 9191-6). Cord blood EPCs or ASC alone or in mixture (in a ratio of 4:1) were suspended in 1.5 mg/ml rat-tail collagen 1,100 ng/ml human fibronectin (Chemicon, Temecula, Calif.), 1.5 mg/ml sodium bicarbonate (Sigma, St. Louis, Mo.), 25 mM HEPES (Cambrex), 10% FBS, 30% EGM-2/F in EBM-2 to the final concentration 2×10⁶ cells/ml. The cell suspensions were placed in a 12-well tissue culture dish (1 ml/well) for 30 minutes at 37° C. for polymerization. The gels were then covered with complete EGM-2/F for overnight incubation. The following day, gels (about 200-500 μl) were implanted subcutaneous on abdominal wall muscle of anesthetized NOD/SCID mice (8-12 weeks old). Each mouse received bilateral implantations of two of the three possible types of the grafts: (1) EPC alone, (2) ASC alone, (3) EPC+ASC mixture, which were randomly arranged between the mice (one graft in each of the flanks). At specific timepoints post-transplantation, the grafts were excised and preserved in 10% formalin, paraffin embedded and evaluated by immunohistochemical evaluation.

In the set of experiments addressing the role of PDGF-BB in EPC-ASC vessel assembly, 10 ng/ml of neutralizing anti-human PDGF-BB IgGs or isotype control goat IgGs (RnD Systems,) were added to the cell/gel mixture prior to polymerization.

Implantation of Cells into Ischemic Myocardium

A myocardial infarction model was created in adult male 300-350 g nude rats (Harlen, Indianapolis, Ind.) as described (Pfeffer, et al., AmJPhysiol 260, H1406-1414 (1991). Animals were anesthetized with 1.5% isoflurane inhalation and a left thoracotomy performed through the fourth intercostals space. The pericardium was opened and the left anterior descending coronary artery ligated permanently with 3-0 silk suture at a site 3 mm distal to the edge of the left atrial appendage. Twenty minutes post-ligation, cell suspension comprised of a total of 1×10⁶ cells (2×10⁵ ASCs and 8×10⁵ EPCs) per 30 ul EGM-2/10% FBS mixed with 70 ul of collagen/fibronectin solution (prepared on ice as above), were injected with a 29 G tuberculin needle directly into left ventricular myocardium, divided among 4-6 sites bordering the ischemic region (25 ul per injection site). After injections, the thorax and muscle were closed with 6-0 silk suture and skin was closed with surgical glue. Cardiac tissue was removed at day 6 following cell implantation, preserved in 10% formalin, paraffin embedded and evaluated by immunohistochemistry.

Immunohistochemical Evaluation of Collagen Plugs

To visualize human endothelial cells, sections were boiled in EDTA Retrieval buffer (20 mm), incubated with 2% H₂O₂ for 10 mm to block endogenous peroxide and incubated with M.O.M. mouse IgGs blocking reagent (Vector, Burlingame, Calif.) for 1 h. Sections were incubated with mouse anti-human CD3I antibodies (LabVision, Fremont Calif.; dilution 1:100), followed by incubation with biotinylated horse anti-mouse IgGs (Vector) for 30 mm.

To visualize human ASCs and host smooth muscle cells, sections were incubated with 2% H₂O₂ for 10 mm to block endogenous peroxide, incubated with M.O.M. mouse

IgGs Blocking Reagent for 1 h, Followed by Incubation with Anti-α-Smooth Muscle Actin

IgGs (αSMA; Sigma, dilution 1:800) for 1 h, followed by incubation with biotinylated horse anti-mouse IgGs (Vector) for 30 mm.

To visualize GFP transduced ASCs, sections were boiled in EDTA Retrieval buffer (20 mm), incubated with 2% H₂O₂ for 10 mm to block endogenous peroxide. Sections were incubated with rabbit anti-GFP IgGs (Clontech, Mountain View, Calif., dilution 1:100) or isotype control rabbit IgGs for 1 h, followed by incubation with biotinylated goat antirabbit IgGs (Vector) for 30 mm.

Antigen-antibody complexes were revealed by incubation with VECTASTAIN® ABC Reagent (HRP) for 30 mm followed by exposure to DAB substrate (Sigma). For immunofluorescent evaluation of endothelial cell with ASC co-assembly, sections were incubated with rabbit anti-factor VIII IgGs (Sigma; dilution 1:200) and mouse anti-SMA

(Sigma, dilution 1:200) for 1 h. To detect primary IgGs sections were incubated with goat anti-rabbit-TRITC (Invitrogen, 1:200) and chicken anti-mouse Alexa 488

(Invitrogen; 1:200) IgG for 30 minutes. The nuclei were counterstained with DAPI

(Sigma).

For immunofluorescent evaluation of endothelial cell with ASC co-localization in the myocardium, sections were incubated with mouse anti-human CD3I (LabVision) and rabbit anti-GFP (Clontech) or with or isotype control mouse and rabbit IgGs for 1 h, with subsequent incubation with horse anti-mouse IgGs (Vector), Streptavidine-Alexa 594 (Invitrogen) and goat anti-rabbit Alexa 488 (Invitrogen), for 30 mm with each reagent. The nuclei were counterstained with DAPI (Sigma). Stained sections were visualized with a Nikon microscope (TE-2000).

Proliferation and Apoptosis Assay

To evaluate proliferation of donor cells in the implants NOD/SCID mice received i.p. injections of 1.5 mg BrdU (Sigma) in saline solution immediately after implantation and every day until sacrifice. Gels were harvested at day 6 and processed for paraffin sectioning as described above. Thin sections were evaluated for BrdU incorporation using the BD BrdU Detection Kit (BD Pharmingen; San Diego, Calif.).

To evaluate rate of donor cell apoptosis, sections prepared from gels harvested on day 14 were processed using the Apoptosis ApopTag Plus Fluorescein In Situ Apoptosis Detection Kit (Chemicon).

Results

Vasculogenesis by Human Primary ASC and EPC

It has been previously reported that human UCB EPCs embedded in a collagen/fibronectin matrix formed perfused, albeit transitory, capillaries when implanted subdermally in immunotolerant mice. To evaluate the potential for ASC to assist in vessel formation and stabilization of neovasculature, studies were conducted as disclosed herein using a collagen/fibronectin matrix containing either: (1) EPCs, (2) ASCs, or (3) a 1:4 mixture of ASCs to EPCs (A+E). A clear difference was found in the appearance of the collagen/fibronectin matrices containing cells when harvested from mice at 2 weeks after implantation. While implants containing EPCs or ASCs alone were whitish in color with superficial, thin vascular structures, matrices containing the combination of the two cell types were consistently red due to the presence of blood filled vessels. Additionally, it was observed that implants containing A+E were tightly associated with the muscle fascia, while implants with either ASCs or EPCs were loosely attached to host tissue.

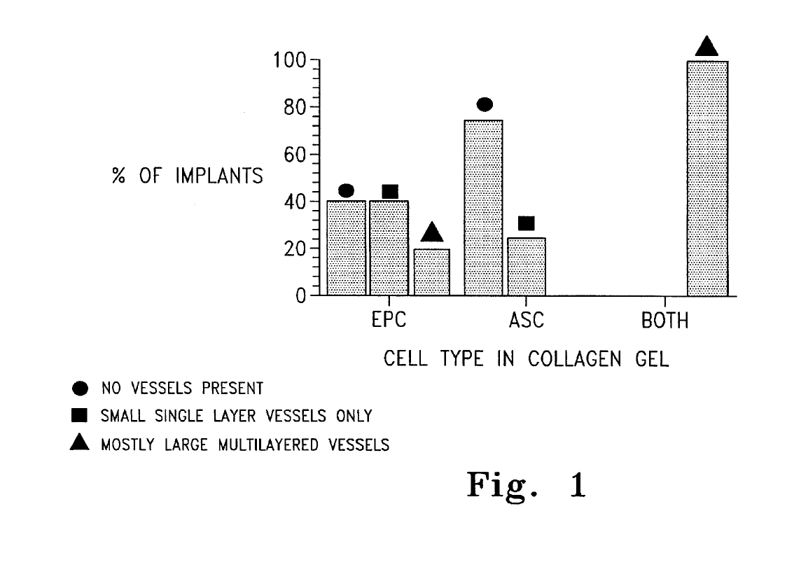

The visible differences in blood content of implants with human ASCs and EPCs indicated that this combination formed an extensive network of vessels that connected with the host vasculature. Microscopic examination of implant sections stained with hematoxylin and eosin, or for endothelial or smooth muscle antigens, was used to identify vessels as luminal structures that were further classified according to their size, presence of single or multiple layers of cells in the vascular wall, and the presence or absence of contained blood elements (FIG. 1). Among implants with EPCs, only 20% contained at least one multilayered vessel, while 40% contained only single layer vessels, and 40% evidenced no vessels. Among implants containing only ASCs, none of the implants contained complex multi-layered vessels, 30% contained small simple vessels, and 70% possessed no visible vessels. Remarkably, all implants containing A+E contained numerous vessels comprised of an endothelial layer surrounded by a layer of mural cells, with connections to the host vasculature evidenced by the presence of erythrocytes within the lumens.

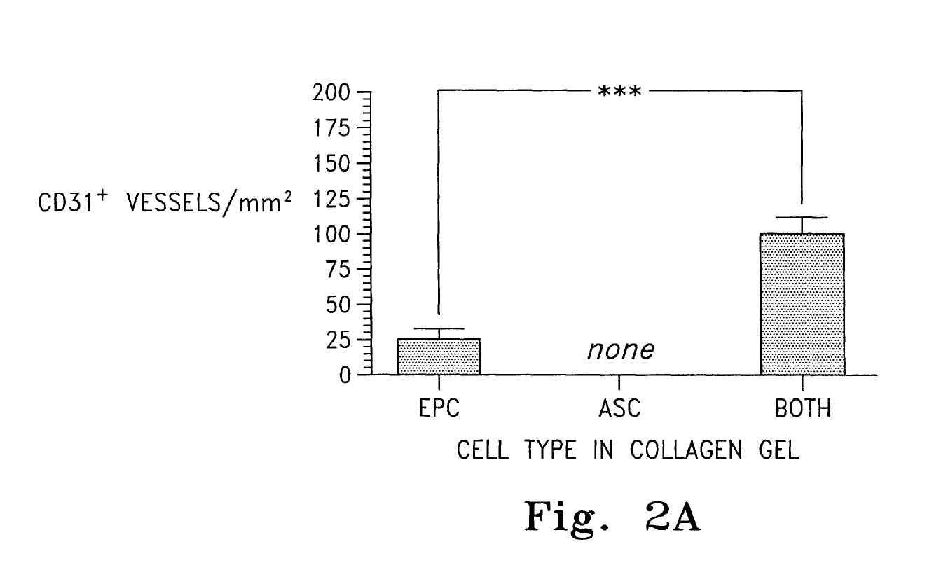

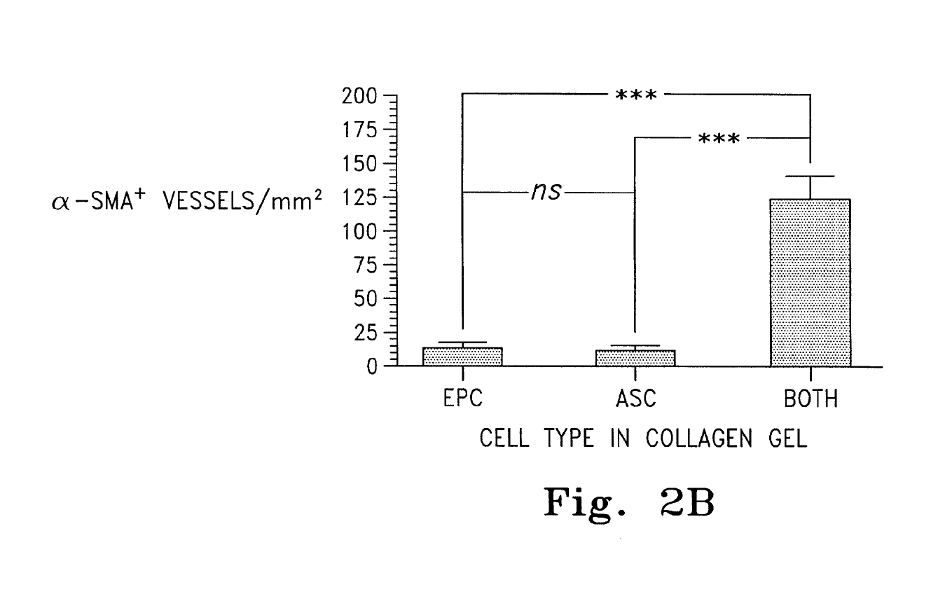

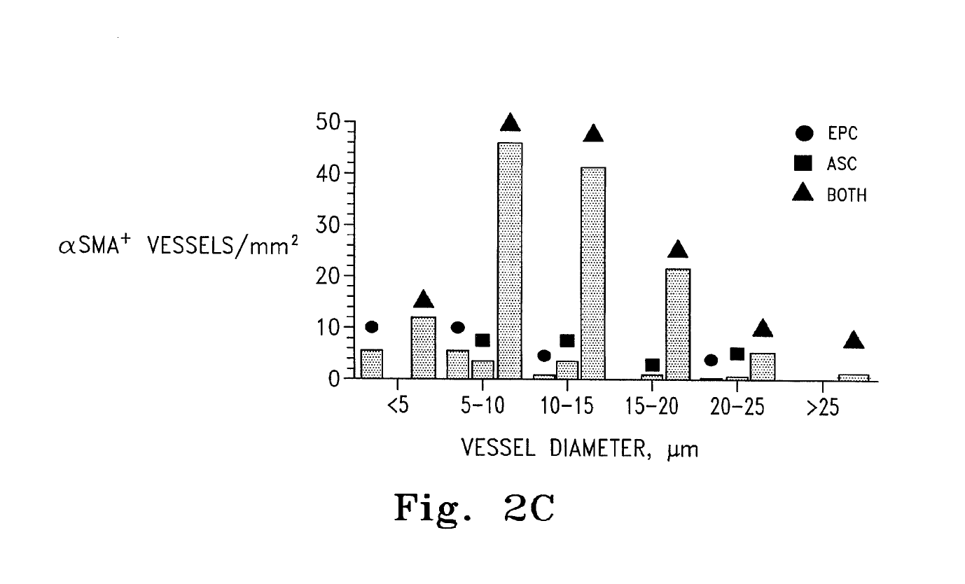

Vessel density and composition in the implants was further assessed by staining for human vascular endothelial cells (human specific CD3I/PECAM) and smooth muscle cells (α-SMA). Vessels containing human endothelial cells or cells staining for α-SMA and possessing distinct lumina were quantitated (FIGS. 2A and 2B). EPC-containing implants gave rise to 26.6±5.8 CD31⁺ and 13.1±3.6 α-SMA⁺ vessels/mm², the latter indicating that host mural cells invaded the implants and contributed to vessel formation.

ASC implants possessed 10.2±3.5 α-SMK vessels/mm², which were presumably derived from the input human ASCs. Vessels containing human CD3I-expressing cells were not detected in any of the implants containing only ASCs, indicating that the observed vessels either incorporated host endothelial cells or were pseudovessels formed by ASCs but lacking an endothelial layer. By comparison to these groups, the A+E implants contained remarkably more vessels as enumerated by both CD31 (122.4±9.8 vessels/mm²) and α-SMA (124.7±19.7 vessels/mm²) staining (p<0.001). The similar density of CD31⁺ and α-SMA⁺ vessels formed by the combination of cells is consistent with routine joint participation of A+E in the neovessels. Analysis of the vascular networks with respect to vessel diameter revealed that the dual cell implants gave rise to a broad distribution of vascular dimension, which did not occur in implants with either cell type alone (FIG. 2C).

To confirm that implants with both A+E formed multilayered vessels, sections were double-stained with antibodies directed against the endothelial marker—factor VIII and against the ASC/mural marker α-smooth muscle actin. Confocal immunofluorescence micrographs of longitudinal and cross-sectional views confirmed bilaminar vessels with an inner endothelial layer surrounded by an outer layer of α-SMA⁺ cells (presumably ASCs). Moreover, the presence of autofluorescent erythrocytes in the lumen was apparent.

To test the origin of the mural layer of the newly formed vessels, experiments were conducted in which ASCs transduced with lentiviral vectors encoding GFP were co-embedded with EPCs and implanted into mice. Immunodetection of GFP at day 14 revealed that vessels were routinely coated by GFP-expressing ASCs, confirming human donor origin of the mural cells of the assembled vessels.

Donor-Derived Neovascular Networks Link to Host Vasculature

It is apparent from the above data that ASCs and EPCs in the matrix operate in concert to assemble a vascular network with a range of diameters in these implants. To determine whether these vessels inosculated with the host vasculature, the CD3-positive vessels which clearly contained erythrocytes were scored at 14 days postimplantation (FIG. 3A). In the implants containing solely EPCs, 3% of the total vessels detected contained erythrocytes, while none were observed in ASC implants. Conversely, nearly 75% (92.5±16.2 per mm²) of CD31⁺ vessels observed in A+E implants were functional and blood-filled, demonstrating connections with host (mouse) vasculature and incorporation into the circulatory system. Microbubble contrast-enhanced ultrasound demonstrated function of the network with flow manifested in implants following systemic injection of microbubbles two weeks post-implantation.

The dynamics of vessel formation in vivo by the combination of A+E were evaluated in implants harvested at 2, 4 and 6 days post-placement. At day two following implantation, endothelial cells had assembled into tubes, which had not formed apparent connections with host vasculature. By day 4, a significant number of the newly formed vessels were filled with erythrocytes (FIG. 3B). A further increase in the density of functional, erythrocyte-containing vessels was observed at 6 days; moreover, the vessels had formed branching networks throughout the implants. Thus, the cooperative formation of vessels by ASCs and EPCs occurs quickly in vivo and is followed by connection with the host vasculature.

Vasculogenesis involves reduction of EPC apoptosis and requires PDGF BrdU labeling was employed to determine the cycling status of cells comprising vessels within the matrices containing A+E. Cells that had undergone DNA synthesis during the 6 days following matrix insertion were observed throughout the implants, with many located in vessel walls in both the luminal (EPCs) and abluminal layer (ASCs).

Implants containing solely EPCs were previously observed to form only transient vessels. Accordingly, ASCs role in preventing vessel regression by affecting apoptosis of endothelial cells was investigated. Matrices containing ASCs and EPCs alone, or A+E were analyzed for apoptotic cells by TUNEL staining at day 14 post-implantation. Many apoptotic cells were observed in matrices implanted with only EPCs. Conversely, implants with only ASCs had few apoptotic cells and importantly, apoptosis was suppressed to very low levels in combination implants.

In vitro interaction of ASCs and endothelial cells is accompanied by secretion of complementary growth factors, including PDGF-BB by endothelial cells. To evaluate whether the in vivo process of vasculogenesis conducted by A+E depended on signaling by PDGF-BB, gels were implanted with the addition of either control or anti-PDGF neutralizing antibodies. The data revealed that the specific disruption of vascular assembly by antagonism of PDGF-BB; while both ASC5 and endothelial cells survive within these gels, their assembly into lumen-containing structures is notably absent.

To evaluate the ability of A+E to conduct vasculogenesis in the context of an ischemic tissue environment, the cells were suspended at a 1:4 ratio in a collagen matrix and injected into rat myocardium following LAD ligation. After 6 days, immunohistochemical analysis of myocardial sections revealed the presence of vessels incorporating human endothelial cells and conducting blood, located in the intramyocardial as well as in the epicardial pen-infarct regions.

1. A composition, comprising a mixture of:
a quantity of endothelial cells; and
a quantity of adipose stromal cells;
wherein when the mixture is applied to a tissue of a patient, the mixture facilitates production of neovasculature to treat the tissue.

2. The composition of claim 1, wherein the quantity of adipose stromal cells are obtained by processing adipose tissue, and wherein the quantity of endothelial cells are obtained by processing non-adipose tissue.

3. The composition of claim 1, wherein the mixture is obtained from a stromal cell fraction.

4. The composition of claim 1, wherein when the tissue comprises ischemic tissue, application of the mixture to the ischemic tissue treats the ischemic tissue and/or prevents further ischemia.

5. The composition of claim 1, wherein the quantity of endothelial cells and the quantity of adipose stromal cells are each purified prior to forming the mixture.

6. The composition of claim 1 wherein the quantity of endothelial cells comprise a quantity of endothelial progenitor cells.

7. The composition of claim 1, wherein the quantity of endothelial progenitor cells are isolated from umbilical cord blood.

8. The composition of claim 1, further comprising:
a substance selected from the group consisting of an extracellular matrix protein, a glycoprotein, and a biocompatible polymer.

9. The composition of claim 1, wherein the mixture comprises at least twice as many endothelial cells than adipose stromal cells.

10. The composition of claim 1, further comprising:
a biocompatible polymer selected from the group consisting of collagen, a peptide, polyglycol acid (PGA), polylactic acid (PLA), and a co-polymer of PGA and PLA.

11. The composition of claim 1, further comprising:
a quantity of collagen.

12. The composition of claim 1, further comprising:
a quantity of fibronectin.

13. The composition of claim 10, wherein when the biocompatible polymer is applied to the tissue with the quantity of endothelial cells and the quantity of adipose stromal cells, the biocompatible polymer forms a matrix capable of retaining at least a majority of the quantity of endothelial cells and the quantity of adipose stromal cells.

14. The composition of claim 1, further comprising:
a substance combined with the mixture, the substance selected from the group consisting of a hydrogel, alginate, collagen, fibronectin, and a peptide hydrogel.

15. The composition of claim 1, further comprising:
a pharmaceutically acceptable carrier;
wherein the mixture is suspended in said carrier.

16. The composition of claim 1, wherein the quantity of endothelial cells and the quantity of adipose stromal cells are each native cell populations.

17. A composition, comprising a mixture of:
a quantity of endothelial cells;
a quantity of adipose stromal cells; and
a biocompatible matrix comprising a substance selected from the group consisting of collagen, a peptide, polyglycol acid (PGA), polylactic acid (PLA), and a co-polymer of PGA and PLA;
wherein when the mixture is applied to a tissue of a patient, the mixture facilitates production of neovasculature to treat the tissue.

18. The composition of claim 17, wherein the quantity of adipose stromal cells are obtained by processing adipose tissue and removing adipocytes therefrom.

19. The composition of claim 17, wherein the quantity of adipose stromal cells comprise pluripotent stem cells expressing at least one cell marker selected from the group consisting of CD14Oa, CD14Ob, and NG2.

20. A composition, comprising a mixture of:
a quantity of endothelial cells isolated from umbilical cord blood;
a quantity of adipose stromal cells isolated from adipose tissue; and
a biocompatible polymer capable of forming a matrix to retain at least a majority of the quantity of endothelial cells and the quantity of adipose stromal cells therein;
wherein when the mixture is applied to an ischemic tissue of a patient, the mixture facilitates production of neovasculature to treat the ischemic tissue and/or to prevent further ischemia.
